(19) United States
(12) Patent Application Publication
(10) Pub. No.: US 2010/0094230 A1
(43) Pub. Date: Apr. 15, 2010

(54) POLYMER MOLDING COMPOUNDS CONTAINING PARTIALLY NEUTRALIZED AGENTS

(75) Inventors: Ralf Dujardin, Neuss (DE); Achim Bertsch, Koeln (DE); Heinz Pudleiner, Krefeld (DE)

Correspondence Address: Baker Donelson Bearman, Caldwell & Berkowitz, PC, 555 Eleventh Street, NW, Sixth Floor, Washington, DC, 20004, US

(73) Assignee: Bayer Innovation GmbH, Duesseldorf (DE)

(21) Appl. No.: 12/526,380

(22) PCT Filed: Jan. 30, 2008
(86) PCT No.: PCT/EP2008/000693

§ 371 Date: Aug. 7, 2009

(30) Foreign Application Priority Data
Feb. 17, 2007 (DE) 102007006761.7

(51) Int. Cl. A61M 25/00; C08K 5/3462

(52) U.S. Cl. 604/265; 523/122

(57) ABSTRACT

The invention relates to polymer molding compositions rendered antibacterial, antiprotozoic, or antimycotic by using partially neutralized active ingredients, to processes for their production, and to their use in moldings, in particular in medical items.

The invention relates to polymer molding compositions rendered antibacterial, antiprotozoic, or antimycotic by using partially neutralized active ingredients, to processes for their production, and to their use in moldings, in particular in medical items.

Polymeric organic materials have become essential in everyday life. Under various conditions in which they are used, workpieces composed of organic materials are naturally susceptible to colonization by microorganisms of a very wide variety of types, examples being bacteria, viruses, or fungi. This colonization poses hygienic and medical risks for the environment of the workpiece, and also for the functioning of the workpiece itself, the latter applying in the event of undesired microbiological degradation of the material.

In particular, the use of polymeric materials for diagnostic and therapeutic purposes has led to a significant and dramatic advance in the technology of modern medicine. On the other hand, the frequent use of said materials in medicine has led to a drastic rise in what are known as foreign-body infections or polymer-associated infections.

Alongside complications of trauma and of thromboembolism, catheter-associated infections extending as far as sepsis are a serious problem with the use of central venous catheters in intensive-care medicine.

Numerous studies have shown that coagulase-negative staphylococci, the transient microbe Staphylococcus aureus, Staphylococcus epidermis and various Candida species are the main causes of catheter-associated infections. During application of the catheter, these microorganisms, which are ubiquitously present on the skin, penetrate the physiological barrier of the skin and thus reach the subcutaneous region and eventually the bloodstream. Adhesion of the bacteria to the plastics surface is regarded as an essential step in the pathogenesis of foreign-body infections. Adhesion of the cutaneous organisms to the polymer surface is followed by the start of metabolically active proliferation of the bacteria with colonization of the polymer. This is associated with production of a biofilm through bacterial excretion of extracellular glycocalix.

The biofilm also assists adhesion of the pathogens and protects them from attack by certain cells of the immune system. In addition, the film forms a barrier impenetrable to many antibiotics. Extensive proliferation of the pathogenic microbes on the polymer surface may finally be followed by septic bacteriaemia. Therapy of such infections requires removal of the infected catheter because chemotherapy with antibiotics would require unphysiologically high doses.

The incidence of bacterially induced infections with central venous catheters averages about 5%. Overall, central venous catheters prove to be responsible for about 90% of all cases of sepsis in intensive care. The use of central venous catheters therefore not only involves a high risk of infection for the patients but also causes extremely high follow-up therapy costs (subsequent treatment, extended stays in clinics).

Pre-, peri- or post-operative measures (e.g. hygiene measures, etc.) are only a partial solution to these problems. A rational strategy for inhibition of polymer-associated infections consists in the modification of the polymeric materials used. The aim of this modification has to be inhibition of adhesion of bacteria and of proliferation of existing adherent bacteria, for causal inhibition of foreign-body infections. By way of example, this can be achieved by incorporating a suitable chemotherapeutic agent into the polymer matrix (e.g. antibiotics), provided that the incorporated active ingredient can also diffuse out of the polymer matrix. In this case, it is possible to extend the release of the antibiotic over a prolonged period, and thus inhibit for a correspondingly prolonged period the processes of adhesion of bacteria and their proliferation on the polymer.

There are previously known methods for preparation of antimicrobially modified polymers. The microbicides here are applied onto the surface or onto a surface layer or introduced into the polymeric material. The following techniques have been described for thermoplastic polyurethanes, which are particularly used for medical applications:
a) adsorption on the polymer surface (passively or via surfactants)
b) introduction into a polymer coating which is applied on the surface of a molding
c) incorporation into the bulk phase of the polymeric substrate material
d) covalent bonding to the polymer surface
e) mixing with a polyurethane-forming component prior to the reaction to give the finished polymer.

By way of example, EP 0 550 875 B1 discloses a process for introducing active ingredients into the outer layer of medical items (impregnation). In this process, the implantable apparatus composed of polymeric material is swollen in a suitable solvent. This alters the polymer matrix to the extent that it becomes possible for a pharmaceutical active ingredient or an active ingredient combination to penetrate into the polymeric material of the implant. Once the solvent has been removed, the active ingredient becomes included within the polymer matrix. After contact with the physiological medium, the active ingredient present in the implantable apparatus is in turn released via diffusion. The release profile here can be adjusted within certain limits via the selection of the solvent and via variation of the experimental conditions.

Polymer materials which are intended for medical applications and which have coatings comprising active ingredient are mentioned by way of example in U.S. Pat. No. 5,019,096. Processes are described for production of the antimicrobially active coatings, and methods are described for application to the surfaces of medical devices. The coatings are composed of a polymer matrix, in particular of polyurethanes, of silicones, or of biodegradable polymers, and of an antimicrobially active substance, preferably of a synergistic combination of a silver salt with chlorhexidine or with an antibiotic.

EP 927 222 B1 describes the introduction of substances having antithrombic or antibiotic action into the reaction mixture for preparation of a TPU.

WO 03/009879 A1 describes medical products with microbicides in the polymer matrix, where the surface has been modified with biosurfactants. Various techniques can be used to introduce the active ingredients into the polymer. The surfactants serve to reduce adhesion of the bacteria on the surface of the molding.

U.S. Pat. No. 5,906,825 describes polymers, among which are polyurethanes, in which biocides or antimicrobial agents (specific description being exclusively of plant ingredients) have been dispersed, the amount being sufficient to suppress the growth of microorganisms coming into contact with the polymer. This can be optimized via addition of an agent which regulates the migration and/or release of the biocide. Naturally occurring substances such as vitamin E are mentioned. Food packaging is the main application.

Zbl. Bakt. 284, 390-401 (1996) describes improved action over a long period of antibiotics dispersed in a silicone polymer matrix or polyurethane polymer matrix, in comparison with antibiotics applied via a deposition technique to the surface or antibiotics introduced in the vicinity of the surface via a technique involving incipient swelling. Here, the high initial rate of release of the antibiotic from the surface into an ambient aqueous medium is subject to very marked, non-reproducible variations.

U.S. Pat. No. 6,641,831 describes medical products with retarded pharmacological activity, this being controlled via introduction of two substances having different levels of lipophilic properties. The core of the invention is the effect that the release rate of an antimicrobial active ingredient reduces via addition of a more lipophilic substance, the result being that release is maintained over a longer period. It is said to be preferable that the active ingredient does not have high solubility in aqueous media. The disclosure includes the fact that active ingredients can be lipophilized via covalent or non-covalent modifications, such as complexing or salt formation. By way of example, it is said that gentamicin salt or base can be modified with a lipophilic fatty acid.

A factor common to all of the methods mentioned is that an additional operation is required to equip the medical equipment with a pharmacologically active substance, namely either pretreatment of the polymer material prior to processing or posttreatment of the resultant moldings. This incurs additional costs and increases the time consumed in the production process. Further problems of the methods consist in the use of organic solvents, which mostly cannot be removed entirely from the material.

Another factor common to all of the methods mentioned here is that the time-limited long-term action of the antimicrobial modification of the moldings composed of polymeric material, and particularly of medical products, is optimized in use on or in the patient. However, this is not satisfactorily achieved at the same time as avoidance of the risk of initial microbial infection of the molding itself, or of humans or animals via the molding.

The medical products intended here are predominantly used intracorporeally. By way of example, catheters pass through the surface of the body for the entire period of their use, and therefore pose a particularly high risk of microbial infection, as described at an earlier stage above. The risk of initial infection on introduction of the medical products into the body, via microbial contamination, has not yet been adequately reduced by the known antimicrobial modifications.

It is therefore an object, starting from the prior art mentioned, to provide an antimicrobially modified polymer material for the production of medical moldings for implants, in particular catheters, where the long-term action of the material in inhibition of surface colonization by microbes is appropriate for the healing process, and the material here minimizes the risk of initial microbial infection on introduction into the biological tissue, via immediate microbicidal action, and the material has suitable mechanical properties, and its manufacture is simple and advantageous.

Surprisingly, it has now been found that the molding compositions described below of the invention, and the moldings produced therefrom not only exhibit, at the surface, a high initial concentration of active ingredients which inhibits initial colonization by microorganisms on wetting with an aqueous fluid, via a high level of active ingredient release, but also ensure further prolonged active ingredient release at a sufficiently high level for long-term use.

The present invention therefore firstly provides molding compositions comprising at least one thermoplastically processable polymer, in particular thermoplastic polyurethanes (TPUs), copolyesters, and polyether block amides, and also comprising at least one partially neutralized active ingredient.

The present invention secondly provides moldings which comprise molding compositions of the invention.

The active ingredients used in the invention have antibacterial, antiprotozoic or antimycotic, or fungicidal activity, and are therefore considered on the basis of their action to be antibiotics, antiinfectives, antimycotics, or fungicides.

For the purposes of this invention, a partially neutralized active ingredient is either an active ingredient having basic functionality which has been partially neutralized with an acid, or an active ingredient having acidic functionality which has been partially neutralized with a base. The terms basic or acidic functionality, and also acid and base, encompass the well-known terms with the meaning of proton acceptor and proton donor, according to Brönstedt.

For the purposes of this invention, other active ingredients also understood to be a partially neutralized active ingredient are those simultaneously having basic and acidic functionalities, examples being betaines and zwitterions having quaternary nitrogen. In this case, the acidic functionality is partially neutralized with a base, and the basic functionality is partially neutralized with an acid.

Active ingredients suitable in the invention and having basic functionality are organochemical aliphatic and cyclic, in particular heterocyclic, compounds which, by way of example, bear a nitrogen functionality as substituent or within the chain or the ring. Preference is given to active ingredients such as β-lactam antibiotics, examples being penicillins, in particular esters of 6-aminopenicillinic acid, for example bacampicillin, and cephalosporins, in particular cefotiam, esters of 7-aminocephalosporanic acid, e.g. cefpodoxim-proxetil and cefetamet-pivoxil, gyrase-inhibitor antiinfectives, e.g. derivatized quinolones, in particular carboxylic-acid-function-derivatized fluoroquinolonecarboxylic acid derivatives, aminoglycoside antibiotics, e.g. in particular streptomycin, neomycin, gentamicin, tobramycin, netylmycin, and amikacin, tetracycline antibiotics, e.g. in particular docycycline and minocycline, chloramphenicol and derivatives, in particular in the form of monosodium salt of ester of succinic acid, macrolide antibiotics, such as desosamine macrolides, in particular erythromycin, clarithromycin, roxithromycin, azithromycin, erythromycyclamine, dirithromycin, and esters of these, e.g. ketolides, lincosamide antibiotics, e.g. in particular lincomycin and clindamycin, oxazolidinone antibiotics, sulfonamide antimicrobiotics, e.g. in particular sulfisoxazole, sulfadiazine, sulfamethoxazole, sulfamethoxydiazine, sulfalene, and sulfadoxine, diaminopyrimidine antimicrobiotics, e.g. in particular trimethoprim, pyrimethamine, basic ansamycin antibiotics, e.g. in particular rifampicin and rifabutin, and also azole antimycotics, such as imidazole derivatives, e.g. in particular bifonazole, clotrimazole, econazole, miconazole, and isoconazole, and triazole derivatives, e.g. in particular itraconazole, and voriconazole.

Active ingredients suitable according to the invention having acidic functionality are organochemical aliphatic and cyclic, in particular heterocyclic, compounds having substitution by way of example with one or more carboxy groups and/or a sulfo group. Preference is given to active ingredients such as β-lactam antibiotics, examples being penicillins, in particular 6-aminopenicillanic acids, for example penicillin G, propicillin, amoxicillin, ampicillin, mezlocillin, oxacillin, and flucloxacillin, and clavulanic acid, and cephalosporins, in particular substituted 7-aminocephalosporanic acids, e.g. cefazolin, cefuroxim, cefoxitin, cefotetan, cefotaxim, and ceftriaxon, and oxacephems, such as latamoxef and flomoxef, and carbapenems, in particular imipenem, and monobactams, in particular aztreonam, and also gyrase inhibitor antiinfectives, such as nalixidic acid and nalixidic acid derivatives, in particular nalixidic acid itself, and also fusidic acid.

Active ingredients suitable according to the invention having betaine structure or zwitterion structure are by way of example cephalosporins, in particular cefotiam and those of the cefalexin group, such as cefaclor, those of the ceftazidim group, such as ceftazidim, cefpirom, and cefepim, carbapenems, in particular meropenems, quinolonecarboxylic acids, in particular substituted 6-fluoro-1,4-dihydro-4-oxo-7-(1-piperazinyl)quinoline-3-carboxylic acids, e.g. norfloxacin, ciprofloxacin, ofloxacin, sparfloxacin, grepafloxacin, and enrofloxacin, substituted 6-fluoro-1,4-dihydro-4-oxo-7-(1-pyrrolidine)quinoline-3-carboxylic acids, e.g. clinafloxacin, moxifloxacin, and trovafloxacin, and also cyclic peptide antibiotics, such as glycopeptides, e.g. in particular vancomycin and teicoplanin, and streptogramins, e.g. in particular pristinamycins.

Very particularly preferred active ingredients are norfloxacin, ciprofloxacin, clinafloxacin, and moxifloxacin.

According to the invention, the partially neutralized active ingredients can also be used in the form of active ingredient combinations in the moldings, and these combinations include those with structurally or functionally different and/or with non-neutralized active ingredients from the substance classes used according to the invention, as long as their actions are not antagonistic.

Acids that can be used according to the invention are generally any of the familiar inorganic or organic acids or proton donors. Examples of those used are, as a function of the basicity and stability of the active ingredient to be partially neutralized, and also, in the case of medical applications, of the level of physiological tolerance, mineral acids, mono-, di-, tri-, and polyfunctional aliphatic and aromatic carboxylic acids, and hydroxycarboxylic acids; a polybasic carboxylic acid here can have been partially esterified with short- and long-chain alcohols, and hydroxycarboxylic acids can have been esterified with carboxylic acids, and hydroxycarboxylic acids can have been esterified with glycosidically bonded carbohydrates, acidic amino acids, sulfonic acids, e.g. in particular aliphatic perfluorosulfonic acids, and phenols. Compounds that can be used with preference are hydrogen chloride, sulfuric acid, phosphoric acid, phosphoric mono- and diesters, acetic acid, stearic acid, palmitic acid, malonic acid, succinic acid, glutaric acid, adipic acid, malic acid, tartaric acid, ethyl hydrogensuccinate (succinic monoester), citric acid, acetylsalicylic acid, glutamic acid, and perfluorobutanesulfonic acid. Hydrogen chloride is used with particular preference.

Bases that can be used according to the invention are generally any of the familiar inorganic or organic proton acceptors. Examples of those used are, as a function of the acidity and stability of the active ingredient to be partially neutralized, and also, in the case of medical applications, of the level of physiological tolerance, alkali metal hydrides, alkali metal alkoxides, alkali metal carbonates, alkaline earth metal carbonates, alkali metal hydrogencarbonates, alkaline earth metal hydrogencarbonates, and nitrogen bases, e.g. primary, secondary, and tertiary aliphatic, cycloaliphatic, and aromatic amines. Compounds that can be used with preference are sodium hydride, sodium methoxide, sodium hydroxide, magnesium hydroxide, calcium hydroxide, sodium carbonate and potassium carbonate, sodium hydrogencarbonate and potassium hydrogencarbonate, triethylamines, dibenzylamines, diisopropylamines, pyridine, quinoline, diazabicyclooctane (DABCO), diazabicyclononene (DBN), and diazabicycloundecene (DBU).

According to the invention, the active ingredients having betaine structure or zwitterion structure can be partially neutralized either with acids or with bases, for example those from the lists given above.

The partial neutralization can take place within a wide range of equivalence. By way of example, from 0.01 to 0.95 equivalent of acid is used per equivalent of basic functionality in the active ingredient, or from 0.01 to 0.95 equivalent of base is used per equivalent of acidic functionality in the active ingredient. It is preferable to use from 0.01 to 0.95 equivalent, particularly preferably from 0.2 to 0.8 equivalent, of acid or base per mole of active ingredient.

One particularly preferred embodiment of the invention uses quinolone antiinfectives, particularly preferably ciprofloxacin, neutralized with from 0.1 to 0.9 mol of hydrogen chloride per mole of active ingredient.

The neutralization of the active ingredients for the use according to the invention in the polymer takes place by the well-known traditional or more recent methods of organic chemistry. By way of example, therefore, the active ingredient can be suspended or dissolved in a suitable solvent, and the acid or base in undiluted or dissolved form can be added to this mixture. The partially neutralized active ingredient can then be obtained by crystallization or by evaporation of the solvent. However, it is also possible to carry out the neutralization by means of an adsorbent, such as silica gel or aluminum oxide, loaded with the relevant acid or base, or by means of an anionic or cationic ion exchanger. The general method is analogous with column chromatography, via dissolution of the active ingredient in a suitable solvent as mobile phase and continuous discontinuous contact with the stationary phase loaded with the acid or base.

It is also possible here in principle to delay obtaining the partially neutralized active ingredient until a second step, by mixing the equimolar neutralized active ingredient with non-neutralized active ingredient. This can take place in homogeneous solution and/or in liquid form, or else in solid form, for example in crystalline or amorphous powder form.

The partially neutralized active ingredient used must have adequate (chemical) stability in the polymer matrix. Furthermore, no impairment of the microbiological activity of the active ingredient in the polymer matrix is permitted under the conditions of the incorporation process, and the active ingredient must therefore have adequate stability at the temperatures and residence times required for the thermoplastic processing of the polymeric material: from 150 to 200° C. and from 2 to 5 min.

The incorporation of the pharmaceutically active substance should not impair either the biocompatibility of the polymer surface or other desirable polymer-specific properties of the polymeric material (elasticity, ultimate tensile strength, etc.).

The active ingredients are preferably incorporated at a concentration appropriate to their activity. The proportion of active ingredient (calculated as non-neutralized active ingredient) in the molding composition is preferably in the range from 0.1 to 5.0% by weight, particularly preferably from 0.5 to 2% by weight, based in each case on the molding composition. It is very particularly preferable to use from 1 to 2% by weight of ciprofloxacin.

Particularly suitable thermoplastically processable polymers are thermoplastic polyurethanes, polyether block amides, and copolyesters, preferably thermoplastic polyurethanes and polyether block amides, and particularly preferably thermoplastic polyurethanes.

The thermoplastically processable polyurethanes that can be used according to the invention are obtainable via reaction of the following polyurethane-forming components:

A) organic diisocyanate,
B) linear hydroxy-terminated polyol whose molecular weight is from 500 to 10 000,
C) chain extender whose molecular weight is from 60 to 500,
where the molar ratio of the NCO groups in A) to the groups reactive towards isocyanate in B) and C) is from 0.9 to 1.2.

Examples of organic diisocyanates A) that can be used are aliphatic, cycloaliphatic, heterocyclic and aromatic diisocyanates, as described in Justus Liebigs Annalen der Chemie, 562, pp. 75-136. Aliphatic and cycloaliphatic diisocyanates are preferred.

Individual compounds which may be mentioned by way of example are: aliphatic diisocyanates, such as hexamethylene diisocyanate, cycloaliphatic diisocyanates, such as isophorone diisocyanate, cyclohexane 1,4-diisocyanate, 1-methylcyclohexane 2,4-diisocyanate and 1-methylcyclohexane 2,6-diisocyanate, and also the corresponding isomer mixtures, dicyclohexylmethane 4,4′-diisocyanate, dicyclohexylmethane 2,4′-diisocyanate and dicyclohexylmethane 2,2′-diisocyanate, and also the corresponding isomer mixtures, aromatic diisocyanates, such as tolylene 2,4-diisocyanate, mixtures composed of tolylene 2,4-diisocyanate and tolylene 2,6-diisocyanate, diphenylmethane 4,4′-diisocyanate, diphenylmethane 2,4′-diisocyanate and diphenylmethane 2,2′-diisocyanate, mixtures composed of diphenylmethane 2,4′-diisocyanate and diphenylmethane 4,4′-diisocyanate, urethane-modified liquid diphenylmethane 4,4′-diisocyanate and diphenylmethane 2,4′-diisocyanate, 4,4′-diisocyanato-(1,2)-diphenylethane and naphthylene 1,5-diisocyanate. It is preferable to use hexamethylene 1,6-diisocyanate, isophorone diisocyanate, dicyclohexylmethane diisocyanate, diphenylmethane diisocyanate isomer mixtures with >96% by weight content of diphenylmethane 4,4′-diisocyanate and in particular diphenylmethane 4,4′-diisocyanate and naphthylene 1,5-diisocyanate. The diisocyanates mentioned may be used individually or in the form of mixtures with one another. They can also be used together with up to 15% by weight (based on the total amount of diisocyanate) of a polyisocyanate, for example with triphenylmethane 4,4′,4″-triisocyanate or with polyphenyl polymethylene polyisocyanates.

The component B) used comprises linear hydroxy-terminated polyols whose average molecular weight Mn is from 500 to 10 000, preferably from 500 to 5000, particularly preferably from 600 to 2000. As a consequence of the production process, these often comprise small amounts of branched compounds. A term often used is therefore “substantially linear polyols”. Preference is given to polyetherdiols, polycarbonatediols, sterically hindered polyesterdiols, hydroxy-terminated polybutadienes, and mixtures of these.

Other soft segments that can be used comprise polysiloxanediols of the formula (I)

HO—(CH₂)ₙ—[Si(R¹)₂—O—]ₘSi(R¹)₂—(CH₂)ₙ—OH (I)

where
R^{l} is an alkyl group having from 1 to 6 carbon atoms or a phenyl group,
m is from 1 to 30, preferably from 10 to 25 and particularly preferably from 15 to 25, and
n is from 3 to 6,
and these can be used alone or in a mixture with the abovementioned diols. These are known products and can be prepared by synthesis methods known per se, for example via reaction of a silane of the formula (II)

H—[Si(R¹)₂—O—]ₘSi(R¹)₂—H (II)

where R^{l} and m are as defined above,
in a ratio of 1:2 with an unsaturated, aliphatic or cycloaliphatic alcohol, e.g. allyl alcohol, buten-(1)-ol or penten-(1)-ol in the presence of a catalyst, e.g. hexachloroplatinic acid.

Suitable polyetherdiols can be prepared by reacting one or more alkylene oxides having from 2 to 4 carbon atoms in the alkylene radical with a starter molecule which contains two active hydrogen atoms in bonded form. Examples of alkylene oxides that may be mentioned are: ethylene oxide, propylene 1,2-oxide, epichlorohydrin and butylene 1,2-oxide and butylene 2,3-oxide. It is preferable to use ethylene oxide, propylene oxide and mixtures composed of propylene 1,2-oxide and ethylene oxide. The alkylene oxides can be used individually, or in alternating succession, or in the form of mixtures. Examples of starter molecules that can be used are: water, amino alcohols, such as N-alkyldiethanolamines, e.g. N-methyldiethanolamine, and diols, such as ethylene glycol, propylene 1,3-glycol, 1,4-butanediol and 1,6-hexanediol. Mixtures of starter molecules can also be used, if appropriate. Other suitable polyetherdiols are the tetrahydrofuran-polymerization products containing hydroxy groups. It is also possible to use proportions of from 0 to 30% by weight, based on the bifunctional polyethers, of trifunctional polyethers, their amount being, however, no more than that giving a thermoplastically processable product. The substantially linear polyetherdiols can be used either individually or else in the form of mixtures with one another.

Examples of suitable sterically hindered polyesterdiols can be prepared from dicarboxylic acids having from 2 to 12 carbon atoms, preferably from 4 to 6 carbon atoms, and from polyhydric alcohols. Examples of dicarboxylic acids that can be used are: aliphatic dicarboxylic acids, such as succinic acid, glutaric acid, adipic acid, suberic acid, azelaic acid and sebacic acid and aromatic dicarboxylic acids, such as phthalic acid, isophthalic acid and terephthalic acid. The dicarboxylic acids can be used individually or in the form of mixtures, e.g. in the form of a mixture of succinic, glutaric and adipic acid. To prepare the polyesterdiols it can, if appropriate, be advantageous to use, instead of the dicarboxylic acids, the corresponding dicarboxylic acid derivatives, such as dicarboxylic esters having from 1 to 4 carbon atoms in the alcohol radical, carboxylic anhydrides, or carbonyl chlorides. Examples of polyhydric alcohols are sterically hindered glycols having from 2 to 10, preferably from 2 to 6, carbon atoms, and bearing at least one alkyl moiety in the beta position with respect to the hydroxy group, examples being 2,2-dimethyl-1,3-propanediol, 2-methyl-2-propyl-1,3-propanediol, 2,2-diethyl-1,3-propanediol, 2-ethyl-1,3-hexanediol, 2,5-dimethyl-2,5-hexanediol, 2,2,4-trimethyl-1,3-pentanediol, or mixtures with ethylene glycol, diethylene glycol, 1,4-butanediol, 1,5-pentanediol, 1,6-hexanediol, 1,10-decanediol, 1,3-propanediol and dipropylene glycol. Depending on the properties required, the polyhydric alcohols can be used alone or, if appropriate, in a mixture with one another. Other suitable compounds are esters of carbonic acid with the diols mentioned, in particular those having from 3 to 6 carbon atoms, examples being 2,2-dimethyl-1,3-propanediol or 1,6-hexanediol, condensates of hydroxycarboxylic acids, such as hydroxycaproic acid, and polymerization products of lactones, for example of unsubstituted or substituted caprolactones. Polyesterdiols preferably used are neopentyl glycol polyadipates and 1,6-hexanediol neopentyl glycol polyadipates. The polyesterdiols can be used individually or in the form of mixtures with one another.

Chain extenders C) used comprise diols, diamines or amino alcohols whose molecular weight is from 60 to 500, preferably aliphatic diols having from 2 to 14 carbon atoms, e.g. ethanediol, 1,6-hexanediol, diethylene glycol, dipropylene glycol and in particular 1,4-butanediol. However, other suitable compounds are diesters of terephthalic acid with glycols having from 2 to 4 carbon atoms, e.g. bis(ethylene glycol) terephthalate or bis(1,4-butanediol) terephthalate, hydroxyalkylene ethers of hydroquinone, e.g. 1,4-di(hydroxyethyl)hydroquinone, ethoxylated bisphenols, (cyclo)aliphatic diamines, e.g. isophoronediamine, ethylenediamine, 1,2-propylenediamine, 1,3-propylenediamine, N-methyl-1,3-propylene diamine, 1,6-hexamethylenediamine, 1,4-diaminocyclohexane, 1,3-diaminocyclohexane, N,N′-dimethylethylenediamine and 4,4′-dicyclohexylmethanediamine and aromatic diamines, e.g. 2,4-tolylenediamine and 2,6-tolylenediamine, 3,5-diethyl-2,4-tolylenediamine and 3,5-diethyl-2,6-tolylenediamine and primary mono-, di-, tri- or tetraalkyl-substituted 4,4′-diaminodiphenylmethanes or amino alcohols, such as ethanolamine, 1-aminopropanol, 2-aminopropanol. It is also possible to use mixtures of the abovementioned chain extenders. Alongside these, it is also possible to add relatively small amounts of crosslinking agents of functionality three or greater, for example glycerol, trimethylolpropane, pentaerythritol, sorbitol. It is particularly preferable to use 1,4-butanediol, 1,6-hexanediol, isophoronediamine and mixtures of these.

It is also possible to use small amounts of conventional monofunctional compounds, for example as chain terminators or mold-release agents. By way of example, mention may be made of alcohols, such as octanol and stearyl alcohol, or amines, such as butylamine and stearylamine.

The molar ratios of the structural components can be varied over a wide range, thus permitting adjustment of the properties of the product. Molar ratios of polyols to chain extenders of from 1:1 to 1:12 have proven successful. The molar ratio of diisocyanates and polyols is preferably from 1.2:1 to 30:1. Ratios of from 2:1 to 12:1 are particularly preferred. To prepare the TPUs, the amounts of the structural components reacted, if appropriate in the presence of catalysts, of auxiliaries and of additives, can be such that the ratio of equivalents of NCO groups to the total of the NCO-reactive groups, in particular of the hydroxy or amino groups of the lower-molecular-weight diols/triols, and amines and of the polyols is from 0.9:1 to 1.2:1, preferably from 0.98:1 to 1.05:1, particularly preferably from 1.005:1 to 1.01:1.

The polyurethanes that can be used according to the invention can be prepared without catalysts; in some cases, however, it can be advisable to use catalysts. The amounts generally used of the catalysts are up to 100 ppm, based on the total amount of starting materials. Suitable catalysts according to the invention are the conventional tertiary amines known from the prior art, e.g. triethylamine, dimethylcyclohexylamine, N-methylmorpholine, N,N′-dimethylpiperazine, 2-(dimethylaminoethoxy)ethanol, diazabicyclo[2.2.2]octane and the like, and also in particular organometallic compounds, such as titanic esters, iron compounds, tin compounds, e.g. stannous diacetate, stannous dioctoate, stannous dilaurate or the dialkyltin salts of aliphatic carboxylic acids. Dibutyltin diacetate and dibutyltin dilaurate are preferred. Amounts of from 1 to 10 ppm of these are sufficient to catalyze the reaction.

Alongside the TPU components and the catalysts, it is also possible to add other auxiliaries and additives. By way of example, mention may be made of lubricants, such as fatty acid esters, metal soaps of these, fatty acid amides and silicone compounds, antiblocking agents, inhibitors, stabilizers with respect to hydrolysis, light, heat and discoloration, flame retardants, dyes, pigments, inorganic or organic fillers and reinforcing agents. Reinforcing agents are in particular fibrous reinforcing agents, such as inorganic fibres, which are produced according to the prior art and can also have been sized. Further details concerning the auxiliaries and additives mentioned are found in the technical literature, for example J. H. Saunders, K. C. Frisch: “High Polymers”, volume XVI, Polyurethane [Polyurethanes], Parts 1 and 2, Interscience Publishers 1962 and 1964, R. Gächter, H. Müller (Ed.): Taschenbuch der Kunststoff-Additive [Plastics additives handbook], 3rd edition, Hanser Verlag, Munich 1989, or DE-A 29 01 774.

The thermoplastically processable polyurethane elastomers are preferably constructed in steps in what is known as the prepolymers process. In the prepolymers process, an isocyanate-containing prepolymer is formed from the polyol and from the diisocyanate, and in a second step is reacted with the chain extender. The TPUs can be prepared continuously or batchwise. The best-known industrial preparation processes are the belt process and the extruder process.

Examples of polyether block amides suitable according to the invention are those composed of polymer chains composed of repeat units corresponding to the formula I.

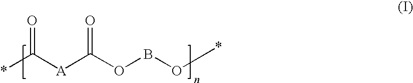

in which
A is the polyamide chain derived from a polyamide having 2 carboxy end groups via loss of the latter, and B is the polyoxyalkylene glycol chain derived from a polyoxyalkylene glycol having terminal OH groups via loss of the latter, and n is the number of units forming the polymer chain. The end groups here are preferably OH groups or moieties of compounds which terminate the polymerization.

The dicarboxylic polyamides having the terminal carboxy groups are obtained in a known manner, for example via polycondensation of one or more lactams or/and of one or more amino acids, or else via polycondensation of a dicarboxylic acid with a diamine, in each case in the presence of an excess of an organic dicarboxylic acid, preferably having terminal carboxy groups. These carboxylic acids become a constituent of the polyamide chain during the polycondensation reaction, and in particular undergo addition at the ends of the same, the product therefore being a polyamide having g-dicarboxylic-acid functionality. The dicarboxylic acid also acts as chain terminator, and it is therefore also used in excess.

The polyamide can be obtained starting from lactams and/or amino acids having a hydrocarbon chain composed of from 4 to 14 carbon atoms, examples being caprolactam, enantholactam, dodecanolactam, undecanolactam, decanolactam, or 11-aminoundecanoic or 12-aminododecanoic acid.

Examples that may be mentioned of polyamides produced via polycondensation of a dicarboxylic acid with a diamine are the condensates composed of hexamethylenediamine and adipic, azelaic, sebacic, and 1,12-dodecanedioic acid, and also the condensates composed of nonamethylenediamine and adipic acid.

Dicarboxylic acids that can be used for the synthesis of the polyamide, i.e. on the one hand for attaching a carboxy group to each end of the polyamide chain, and on the other hand as chain terminator, are those having from 4 to 20 carbon atoms, in particular alkanediacids, such as succinic, adipic, suberic, azelaic, sebacic, undecanedioic, or dodecanedioic acid, or else a cycloaliphatic or aromatic dicarboxylic acid, such as terephthalic or isophthalic acid, or cyclohexane-1,4-dicarboxylic acid.

The polyoxyalkylene glycols having terminal OH groups are unbranched or branched compounds, and have an alkylene moiety having at least 2 carbon atoms. These compounds are in particular polyoxyethylene, polyoxypropylene, and polyoxytetramethylene glycol, and also copolymers thereof.

The average molecular weight of these polyoxyalkylene glycols terminated by OH groups can vary within a wide range, and is advantageously from 100 to 6000, in particular from 200 to 3000.

The proportion by weight of the polyoxyalkylene glycol, based on the total weight of the polyoxyalkylene glycol and dicarboxylic polyamide used for the production of the PEBA polymer, is from 5 to 85%, preferably from 10 to 50%.

Processes for the synthesis of PEBA polymers of this type are known from FR 7 418 913, DE-A 28 02 989, DE-A 28 37 687, DE-A 25 23 991, EP-A 095 893, DE-A 27 12 987, and DE-A 27 16 004.

PEBA polymers preferably suitable according to the invention are those which, in contrast to those described above, have random structure. To produce these polymers, a mixture composed of
1. one or more polyamide-forming compounds from the group of the aminocarboxylic acids or lactams having at least 10 carbon atoms,
2. an α,ω-dihydroxypolyoxyalkylene glycol,
3. at least one organic dicarboxylic acid
   in a ratio by weight 1:(2+3) of from 30:70 to 98:2, where hydroxy groups and carbonyl groups are present in equivalent amounts in (2+3), is heated in the presence of from 2 to 30% by weight of water, based on the polyamide-forming compounds of group 1, under autogenous pressure, at temperatures of from 23° C. to 30° C., and is then further treated after removal of the water, with exclusion of oxygen, at atmospheric pressure or at reduced pressure, at from 250 to 280° C.

Preferred PEBA polymers of this type are described by way of example in DE-A 27 12 987.

Examples of suitable and preferred suitable PEBA polymers are available with trade name PEBAX from Atochem, Vestamid from Hüls AG, Grilamid from EMS-Chemie, and Kellaflex from DSM.

The polyether block amides of the invention, comprising active ingredients, can moreover comprise the additives conventional for plastics. Examples of conventional additives are pigments, stabilizers, flow aids, lubricants, and mold-release agents.

Suitable copolyesters (segmented polyester elastomers) are composed by way of example of a wide variety of repeating short-chain ester units and long-chain ester units, combined via ester bonds, where the short-chain ester units make up about 15-65% by weight of the copolyester, and have the formula (I)

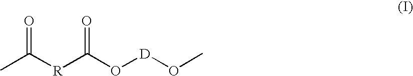

in which R is a divalent dicarboxylic acid moiety whose molecular weight is below about 350, and D is a divalent organic diol moiety whose molecular weight is below about 250; the long-chain ester units make up about 35-85% by weight of the copolyester, and have the formula (II)

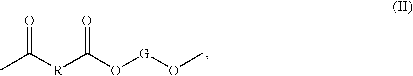

in which
R is a divalent dicarboxylic acid moiety whose molecular weight is below about 350, and G is a divalent long-chain-glycol moiety whose average molecular weight is about 350 to 6000.

The copolyesters that can be used according to the invention can be produced by copolymerizing a) one or more dicarboxylic acids, b) one or more linear, long-chain glycols, and c) one or more low-molecular-weight diols.

The dicarboxylic acids for the production of the copolyester are the aromatic acids having from 8 to 16 carbon atoms, in particular phenylenedicarboxylic acids, such as phthalic, terephthalic, and isophthalic acid.

The low-molecular-weight diols for the reaction to form the short-chain ester units of the copolyesters belong to the classes of the acyclic, alicyclic, and aromatic dihydroxy compounds. The preferred diols have from 2 to 15 carbon atoms, examples being ethylene, propylene, tetramethylene, isobutylene, pentamethylene, 2,2-dimethyltrimethylene, hexamethylene, and decamethylene glycols, dihydroxycyclohexane, cyclohexanedimethanol, resorcinol, hydroquinone, and the like. Among the bisphenols for the present purpose are bis(p-hydroxy)biphenyl, bis(p-hydroxyphenyl)methane, bis(p-hydroxyphenyl)ethane, and bis(p-hydroxyphenyl)propane.

The long-chain glycols for producing the soft segments of the copolyesters preferably have molecular weights of about 600 to 3000. Among these are poly(alkylene ether) glycols in which the alkylene groups have from 2 to 9 carbon atoms.

Other compounds that can be used as long-chain glycol are glycol esters of poly(alkylene oxide)dicarboxylic acids, or polyester glycols.

Among the long-chain glycols are also polyformals obtained via reaction of formaldehyde with glycols. Polythioether glycols are also suitable. Polybutadiene glycols and polyisoprene glycols, copolymers of the same, and saturated hydrogenation products of said materials are satisfactory long-chain polymeric glycols.

Processes for the synthesis of these copolyesters are known from DE-A 2 239 271, DE-A 2 213 128, DE-A 2 449 343, and U.S. Pat. No. 3,023,192.

The copolyesters of the invention comprising active ingredients can moreover comprise the additives conventional for plastics. Examples of conventional additives are lubricants, such as fatty acid esters, metal soaps of these compounds, fatty acid amides, and silicone compounds, antiblocking agents, inhibitors, stabilizers with respect to hydrolysis, light, heat, and discoloration, flame retardants, dyes, pigments, and inorganic or organic fillers and reinforcing agents. Reinforcing agents are in particular fibrous reinforcing materials, e.g. inorganic fibers, which are produced according to the prior art and can also have been treated with a size. Further details concerning the auxiliaries and additives mentioned can be found in the technical literature, for example in J. H. Saunders, K. C. Frisch: “High Polymers”, volume XVI, Polyurethane [Polyurethanes], Parts 1 and 2, Interscience Publishers 1962 or 1964, R. Gächter, H. Müller (ed.): Taschenbuch der Kunststoff-Additive [Plastics additives handbook], 3rd edition, Hanser Verlag, Munich 1989, or DE-A 29 01 774.

The molding compositions of the invention can be produced via extrusion of a melt composed of the polymer and active ingredient. The melt can comprise from 0.01 to 10% by weight, preferably from 0.1 to 5% by weight, of active ingredient. The components can be mixed in any manner using known techniques. By way of example, the active ingredient can be introduced directly in solid form into the polymer melt. It is also possible that a masterbatch comprising active ingredient is directly melted with the polymer, or is mixed with the previously melted polymer. The active ingredient can also be applied to the polymer prior to the melting of the polymer by means of known techniques (via tumbling, spray application, etc.).

The mixing/homogenization of the components can also take place by known techniques by way of kneaders or screw-based machines, preferably in single- or twin-screw extruders, in a temperature range from 150 to 200° C.

The mixing of the components during the extrusion process gives homogeneous dispersion of the active ingredient at the molecular level in the polymer matrix, without any requirement for additional operations.

Studies have shown that homogeneous dispersion of the active ingredient in the polymer matrix is necessary to permit utilization of active ingredient diffusion as adjustable release mechanism. The active ingredient and the polymer should therefore have high physicochemical compatibility. If physicochemical compatibility of active ingredient and polymer is good, the diffusion coefficient of the active ingredient in the polymer is high. The level of release rate of the antibiotic substance can then be regulated via variation of the amount of active ingredient incorporated, since the amount of active ingredient released is then proportional to the concentration in the matrix.

The pellets thus obtained, comprising active ingredient, can be further processed by the known techniques of thermoplastics processing (injection molding, extrusion, etc.). The moldings are speck-free, and flexible, and free from tack, and can be sterilized without difficulty by the familiar processes.

Preferred moldings produced from the molding compositions of the invention are medical products, such as central venous catheters (CVCs), urocatheters, flexible tubing, shunts, cannula, connectors, stoppers, or distributor valves, and particular preference is given to CVCs.

The examples below are intended to illustrate, but not restrict, the invention.

### EXAMPLES

### Example 1

Lenticular pellets (4950 g) of the commercially available aromatic polyether urethane Pellethane 2363-80AE, filled with 20% by weight of barium sulfate, Shore hardness 85 A (Dow Chemical, Midland Mich.) were dried at 80° C. for 24 hours and then intimately mixed with 50 g of ciprofloxacin (betaine), in a gyro-wheel mixer.

### Compounding

This mixture was compounded in a Brabender extruder, composed of:

a 4-zone extruder with twin screws each of diameter (D) 20 mm and of length 25×D; the screw has a devolatilizing section;

a single-aperture round-extrudate die of diameter 3.2 mm;

a water-filled cooling trough of length 2.5 m, temperature about 20° C.;

a differential weigh feeder;

take-off equipment with strand pelletizer.

The above mixture is conveyed by means of the differential weigh feeder into the cold feed barrel of the extruder. The melt is drawn off from the die, and drawn through the cooling trough. The pelletizer provides strand pelletization of the round extrudate.

### Process Parameters:

TABLE 1
Compounding conditions

| Process parameter | | |
|---|---|---|
| Temperature of barrel 1 | 190° | C. |
| Temperature of barrel 2 | 195° | C. |
| Temperature of barrel 3 | 200° | C. |
| Temperature of die | 200° | C. |
| Melt temperature | 210° | C. |
| Melt pressure | 18 | bar |
| Extruder rotation rate | 61 | min⁻¹ |
| Throughput | 4.5 | kg/h |
| Torque | 96 | nM |

The cylindrical pellets, comprising no active ingredient, were extruded in a ZSK twin-screw extruder. The product was a white, homogeneous, speck-free melt, which gave homogeneous cylindrical pellets after cooling in the water/air bath and strand pelletization.

### Injection Molding

An Arburg 270S-500-60 was used, with screw diameter 18 mm. After drying, the pellets were injection molded to give test specimens (plaques, 60×60×2 mm). The parameters selected for this were as follows:

TABLE 2
Injection molding conditions

| Process parameter | | |
|---|---|---|
| Cylinder temperature, heating zone 1 | 190° | C. |
| Cylinder temperature, heating zone 2 | 195° | C. |
| Cylinder temperature, heating zone 3 | 200° | C. |
| Cylinder temperature, heating zone 4 | 200° | C. |
| Mold temperature | 35° | C. |
| Injection pressure | 1600 | bar |
| Rotation rate | 19 | min⁻¹ |
| Hold pressure | 800 | bar |
| Back pressure | 100 | bar |
| Injection time | 0.9 | s |
| Hold pressure time | 10 | s |
| Residual cooling time | 20 | s |
| Feed time | 12.8 | s |
| Cycle time | 37 | s |

For microbiological in-vitro studies, smaller plaques of diameter 5 mm were stamped out from the plaques. These smaller plaques were sterilized using 25 kGr of gamma radiation.

### Example 2

### Comparative Example

Lenticular pellets (4950 g) of the commercially available aromatic polyether urethane Pellethane 2363-80AE, filled with 20% by weight of barium sulfate, Shore hardness 85 A (Dow Chemical, Midland Mich.) were dried at 80° C. for 24 hours and then intimately mixed with 50 g of ciprofloxacin hydrochloride, in a gyro-wheel mixer.

By analogy with the material from example 1, the cylindrical pellets, comprising active ingredient, were extruded in a ZSK twin-screw extruder. The product was a white, speck-free melt, which gave homogeneous cylindrical pellets after cooling in the water/air bath and strand pelletization.

For microbiological in-vitro studies, smaller plaques of diameter 5 mm were stamped out from the plaques. These smaller plaques were sterilized using 25 kGr of gamma radiation.

### Example 3

### Production of Masterbatch for Compounding of the Samples from Examples 7 to 11

Tecothane TT2085A-B20 in the form of commercially available lenticular pellets of size about 2 nun was milled at −40° C. to give a powder, which was then sieved to give two fractions. A 1st fraction with d₅₀=300 μm was used for the examples of the invention.

Ciprofloxacin hydrochloride (d₅₀=9.13 μm) (1000 g) was mixed in an intensive mixer with 2000 g of Tecothane TT2085A-B20 powder (d₅₀=300 μm) comprising no active ingredient. This polymer-active-ingredient powder mixture and a further 2000 g of lenticular Tecothane TT2085A-B20 pellets were fed separately into barrel 1 of the extruder by means of two differential weigh feeders. As in example 1, the cylindrical pellets comprising active ingredient were extruded in a Brabender ZSK twin-screw extruder. The product was a speck-free, white melt which gave cylindrical pellets with 20% by weight of ciprofloxacin hydrochloride after cooling in the water/air bath and strand pelletization.

### Example 4

### Production of Masterbatch for Compounding of the Samples from Examples 12 to 16

Tecothane TT2085A-B20 in the form of commercially available lenticular pellets of size about 2 mm was milled at −40° C. to give a powder, which was then sieved to give two fractions. A 1st fraction with d₅₀=300 μm was used for the examples of the invention.

Ciprofloxacin (betaine) (d₅₀=5.77 μm) (1000 g) was mixed in an intensive mixer with 2000 g of Tecothane TT2085A-B20 powder (d₅₀=300 μm) comprising no active ingredient. This polymer-active-ingredient powder mixture and a further 2000 g of lenticular Tecothane TT2085A-B20 pellets were fed separately into barrel 1 of the extruder by means of two differential weigh feeders. As in example 1, the cylindrical pellets comprising active ingredient were extruded in a Brabender ZSK twin-screw extruder. The product was a speck-free, white melt which gave cylindrical pellets with 20% by weight of ciprofloxacin (betaine) after cooling in the water/air bath and strand pelletization.

### Example 5

The pellets from example 1 were used by an external producer to extrude triple-lumen catheter tubing with external diameter 2 mm comprising ciprofloxacin (betaine).

This catheter tubing was sterilized using 25 kGr of gamma radiation.
The catheter tubing was used in the dynamic model for detection of antimicrobial action of materials, and for determination of elution profile of the incorporated active ingredient.

### Example 6

### Comparative Example

The pellets From example 2 were used by an external producer to extrude triple-lumen catheter tubing with external diameter 2 mm comprising ciprofloxacin hydrochloride.

This catheter tubing was sterilized using 25 kGr of gamma radiation.
The catheter tubing was used in the dynamic model for detection of antimicrobial action of materials, and for determination of elution profile of the incorporated active ingredient d.

### Example 7

### Comparative Example

Masterbatch pellets from example 3 (12.5 g) were mixed in an intensive mixer with 987.5 g of Tecothane TT2085A-B20 pellets comprising no active ingredient. The cylindrical pellets comprising active ingredient were extruded in a Brabender ZSK twin-screw extruder. The product was a homogeneous white melt which, after cooling in the water/air bath and strand pelletization, gave free-flowing cylindrical pellets with 0.25% by weight of ciprofloxacin hydrochloride.

To determine the elution profile of the incorporated active ingredient in the dynamic model for detection of antimicrobial action of materials, extrudate specimens (diameter 2 mm and length about 17 cm) were taken, and the pellets were injection molded to give test specimens (plaques) for the agar diffusion test.

For microbiological in-vitro studies, smaller plaques of diameter 5 mm were stamped out from the plaques. These smaller plaques were sterilized using 25 kGr of gamma radiation.

By analogy with example 7, the following pellets were obtained:

TABLE 3
Constitution of comparative examples 8 to 11

| | | Amount of | Concentration of |
|---|---|---|---|
| | Amount of | Tecothane | ciprofloxacin |
| | masterbatch | TT2085A-B20 | hydrochloride in |
| | from example 3 | pellets comprising | compounded material |
| Example Number | [g] | no active ingredient | after processing |
| Example 8 | 25 | 975 | 0.5% |
| (comparative example) | | | |
| Example 9 | 50 | 950 | 1.0% |
| (comparative example) | | | |
| Example 10 | 75 | 925 | 1.5% |
| (comparative example) | | | |
| Example 11 | 100 | 900 | 2.0% |
| (comparative example) | | | |

### Example 12

Masterbatch pellets from example 4 (12.5 g) were mixed in an intensive mixer with 987.5 g of Tecothane TT2085A-B20 pellets comprising no active ingredient. The cylindrical pellets comprising active ingredient were extruded in a Brabender ZSK twin-screw extruder. The product was a homogeneous white melt which, after cooling in the water/air bath and strand pelletization, gave free-flowing cylindrical pellets with 0.25% by weight of ciprofloxacin (betaine).

To determine the elution profile of the incorporated active ingredient in the dynamic model for detection of antimicrobial action of materials, extrudate specimens (diameter 2 mm and length about 17 cm) were taken, and the pellets were injection molded to give test specimens (plaques) for the agar diffusion test.

For microbiological in-vitro studies, smaller plaques of diameter 5 mm were stamped out from the plaques. These smaller plaques were sterilized using 25 kGr of gamma radiation.

By analogy with example 12, the following pellets were obtained:

TABLE 4
Constitution of examples 13 to 16 of the invention

| | | Amount of | Concentration of |
|---|---|---|---|
| | Amount of | Tecothane | ciprofloxacin |
| | masterbatch | TT2085A-B20 | (betaine) in |
| | from example 4 | pellets comprising | compounded material |
| Example Number | [g] | no active ingredient | after processing |
| Example 13 | 25 | 975 | 0.5% |
| Example 14 | 50 | 950 | 1.0% |
| Example 15 | 75 | 925 | 1.5% |
| Example 16 | 100 | 900 | 2.0% |

### Example 17

The following system of experiments was selected in order to check activity:

### Dynamic Model for Detection of Antimicrobial Action of Materials

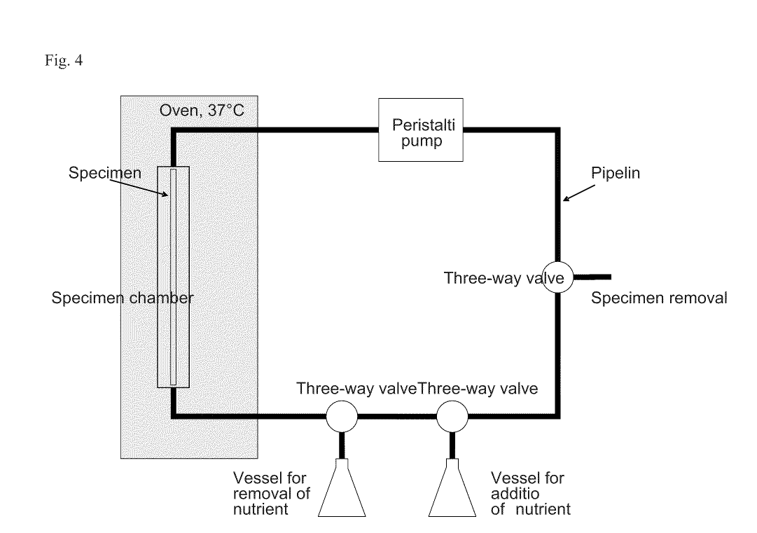

The model described is intended to detect the antimicrobial activity of materials and to demonstrate inhibition of biofilm formation on the materials, and also to record the elution profile of the respective active ingredients from the materials. The experimental apparatus is composed of the following components (cf. also FIGS. 4 and 5):

| | |
|---|---|
| 1. | Reaction chamber |
| 2. | Nutrient replacement system (2 coupled three-way valves) |
| 3. | Specimen chamber |
| 4. | Peristaltic pump |
| 5. | Tubing system |

A piece of extrudate of the specimen to be studied was introduced into a reaction chamber and firmly fixed at both ends by means of shrink tubing. The location of the reaction chamber during the period of the experiment is within the incubator.

The tubing system continues onward to the nutrient replacement system. By using one of the three-way valves, and the outflow position, nutrient can be pumped out from the circuit, and by using the second three-way valve, and the inflow position, nutrient can be introduced into the circuit.

The tubing system continues on by way of the specimen chamber to the system for removal of specimens for determination of number of microbes and addition of the bacterial suspension, and then by way of the peristaltic pump back to the reaction chamber.

### 1. Method

The dynamic biofilm model was used for the studies of the long-term action of the antimicrobial activity of sample specimens (extrudate specimens) and of catheters.

### 1.1. Test Sheets

Mueller-Hinton agar plates were used for the culture mixtures for determination of microbe numbers. For this purpose, 18 ml of Mueller-Hinton agar (Merck KGaA Darmstadt/Batch VM132437 339) were poured into Petri dishes of diameter 9 cm.

### 1.2. Medium

Mueller-Hinton bouillon (Merck KGaA Darmstadt/Batch VM205593 347) was used as medium for the dynamic biofilm model.

### 1.3. Bacterial Suspension

The test strain of Staphylococcus aureus ATTC 29213 was added in the form of suspension in the dynamic biofilm model. A suspension with density corresponding to McFarland 0.5 in NaCl solution at 0.85% strength was prepared from an overnight culture of test strain on Columbia blood agar. A “colony pool” composed of from 3 to 4 colonies applied by spotting with an inoculation loop was used for the suspension. The suspension was diluted twice in a ratio of 1:100. This dilution was used for charging to the model.

### 2.1. Test Mixture

Each separate model circuit (reaction chamber+tubing system) was charged with about 16 ml of medium from its associated supply flask (medium 1.2). 100 μl of the bacterial suspension (1.3) were then added by way of the sampling chamber to the model circuit, using a pipette. In parallel with this, 100 μl of the bacterial suspension were plated out for determination of microbe numbers (1.1).

The average number of microbes present in the model circuit after each addition of the bacterial suspension was at least 200 CPU/ml.

The peristaltic pump was set at a speed of 5 rpm (revolutions per minute), the resultant amount conveyed in the tubing used in the experiment being 0.47 ml/min.

A result was that the content of a model circuit was exchanged and passed over the catheter once in the reaction chamber over the course of a good half hour.

4 ml (25% of the entire liquid) were removed from the model circuit for the first time after 24 hours and then daily or at varying intervals, and replaced by fresh medium.

HPLC was used to determine the ciprofloxacin concentration in the medium removed, and the elution profile was ascertained as a function of time (3.1 elution profile).

The bacterial concentration in each separate model circuit was determined in the specimens removed. 50 μl from the specimen were streaked by an inoculation loop onto a test plate and incubated at 37° C. for 24 hours. The number of microbes was estimated from the growth within the smear, or 50 μl were inoculated with a pipette onto a test plate, and distributed by using a spatula, and incubated at 37° C. for 24 hours, and the calculation was based on colony counting.

In addition to media exchange, 100 μl of the bacterial suspension were added with a pipette to the model circuit daily or in varying intervals by way of the sampling chamber. The number of microbes in the bacterial suspension added varied from 1800 to 15 000 bacteria per ml. Addition of a constant, always identical amount of bacteria was intentionally avoided, since in practice it also has to be expected that there will be varying numbers of pathogens that could come into contact with the catheter.

### 2. Material

### 2.1. Material Specimens

The catheter tubing from examples 5 and 6 was tested to detect antimicrobial action and inhibition of biofilm formation on the materials, and also to determine the elution profile of the respective active ingredients from the catheter tubing.

TABLE 5
Catheter tubing used in the dynamic model

| | | | Concentration |
|---|---|---|---|
| Specimen from | Specimen | Active ingredient | [%] |
| Example 5 | Catheter | Ciprofloxacin | 1 |
| | tubing | (betaine) | |
| Example 6 | Catheter | Ciprofloxacin | 1 |
| (comparative example) | tubing | hydrochloride | |

The extrudate specimens from the examples mentioned below were tested to determine the elution profile of the respective active ingredients

TABLE 6
Extrudate specimens used in the dynamic model

| | | | Concentration |
|---|---|---|---|
| Specimen from | Specimen | Active ingredient | [%] |
| Example 7 | Extrudate | Ciprofloxacin | 0.25 |
| (comparative example) | specimen | hydrochloride | |
| Example 8 | Extrudate | Ciprofloxacin | 0.5 |
| (comparative example) | specimen | hydrochloride | |
| Example 9 | Extrudate | Ciprofloxacin | 1.0 |
| (comparative example) | specimen | hydrochloride | |
| Example 10 | Extrudate | Ciprofloxacin | 1.5 |
| (comparative example) | specimen | hydrochloride | |
| Example 11 | Extrudate | Ciprofloxacin | 2.0 |
| (comparative example) | specimen | hydrochloride | |
| Example 12 | Extrudate | Ciprofloxacin | 0.25 |
| | specimen | (betaine) | |
| Example 13 | Extrudate | Ciprofloxacin | 0.5 |
| | specimen | (betaine) | |
| Example 14 | Extrudate | Ciprofloxacin | 1.0 |
| | specimen | (betaine) | |
| Example 15 | Extrudate | Ciprofloxacin | 1.5 |
| | specimen | (betaine) | |
| Example 16 | Extrudate | Ciprofloxacin | 2.0 |
| | specimen | (betaine) | |

### 2.2. Test Strains

The test strain used for the dynamic biofilm model was a Staphylococcus aureus, strain ATCC 29213, well-known for biofilm formation. The strain was provided by the Medical University in Hanover.

### 3. Evaluation

### 3.1 Elution Profile

TABLE 7
Amount of active ingredient eluted from the
catheter tubing, using specimens taken daily

| | Concentration of ciprofloxacin | Concentration of ciprofloxacin |
|---|---|---|
| | (betaine), detected as hydrochloride | hydrochloride |
| | Catheter tubing composed of | Catheter tubing composed of |
| | material of the invention | material comprising |
| Day on which | comprising ciprofloxacin betaine | ciprofloxacin hydrochloride |
| specimen taken | (example 5) | (example 6) |
| 1st | 1.43 | 2.85 |
| 2nd | 1.55 | 4.85 |
| 3rd | 1.72 | 3.51 |
| 4th | 1.02 | 3.43 |
| 5th | 1.24 | 4.65 |
| 6th | Not determined | Not determined |
| 7th | Not determined | Not determined |
| 8th | 1.86 | 5.87 |
| 9th | 1.54 | 3.87 |
| 10th | 1.01 | 3.28 |
| 11th | 1.29 | 3.53 |
| 12th | Not determined | Not determined |
| 13th | 1.75 | 9.13 |

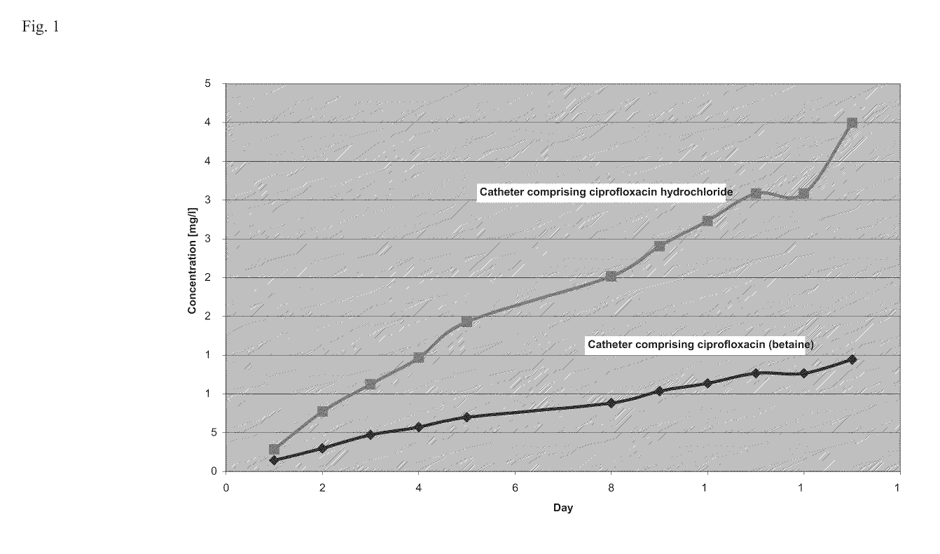

FIG. 1 shows the elution profile as a function of time for the catheter tubing comprising ciprofloxacin hydrochloride from example 6 (comparative example) and for the catheter tubing comprising ciprofloxacin (betaine) (of the invention). The amounts eluted have been totalled.

TABLE 8
Amount of active ingredient eluted from the extrudate specimens
of comparative examples 7 to 11, using specimens taken daily

| Day on | | | | | |
|---|---|---|---|---|---|
| which | Concentration of ciprofloxacin hydrochloride | | | | |
| specimen | | | | | Example |
| taken | Example 7 | Example 8 | Example 9 | Example 10 | 11 |
| 1st | 2.12 | 2.12 | 14.3 | 22.8 | 31.7 |
| 2nd | 1.25 | 3.12 | 7.17 | 12.1 | 17.1 |
| 3rd | 0.95 | 1.99 | 4.74 | 8.56 | 11.0 |
| 4th | 0.96 | 2.17 | 5.11 | 9.26 | 11.82 |
| 5th | 1.21 | 2.03 | 4.03 | 5.86 | 8.08 |
| 6th | 1.02 | 2.49 | 5.85 | 5.66 | 1.99 |
| 7th | 0.62 | 1.34 | 3.27 | 5.03 | 7.03 |
| 8th | 0.5 | 3.07 | 4.77 | 6.51 | 5.27 |
| 9th | 0.73 | 1.68 | 4.14 | 6.34 | 8.33 |
| 10th | 0.92 | 2.29 | 6.04 | 9.09 | 12.52 |
| 11th | 1.5 | 1.25 | 3.23 | 5.24 | 7.32 |
| 12th | 0.5 | 1.25 | 3.05 | 5.12 | 6.75 |
| 13th | 0.73 | 1.68 | 4.14 | 6.34 | 8.33 |

TABLE 9
Amount of active ingredient eluted from the extrudate specimens
of comparative examples 12 to 16, using specimens taken daily

| Day on | Concentration of ciprofloxacin (betaine), | | | | |
|---|---|---|---|---|---|
| which | measured as hydrochloride | | | | |
| specimen | | Example | Example | Example | Example |
| taken | Example 12 | 13 | 14 | 15 | 16 |
| 1st | 1.07 | 0.98 | 2.03 | 19.3 | 80.6 |
| 2nd | 0.88 | 1.03 | 1.35 | 4.87 | 7.74 |
| 3rd | 0.5 | 0.7 | 1.08 | 2.48 | 4.22 |
| 4th | 0.78 | 0.73 | 1.0 | 2.52 | 3.52 |
| 5th | 0.5 | 1.09 | 1.22 | 2.08 | 3.12 |
| 6th | 0.5 | 1.29 | 1.56 | 2087 | 4.07 |
| 7th | 1.05 | 1.11 | 1.69 | 2.63 | 3.33 |
| 8th | 1.27 | 1.23 | 1.57 | 2.08 | 2.64 |
| 9th | 1.11 | 1.17 | 1.77 | 2.81 | 2.72 |
| 10th | Not | Not | Not | Not | Not |
| | deter- | deter- | deter- | deter- | deter- |
| | mined | mined | mined | mined | mined |
| 11th | Not | Not | Not | Not | Not |
| | deter- | deter- | deter- | deter- | deter- |
| | mined | mined | mined | mined | mined |
| 12th | Not | Not | Not | Not | Not |
| | deter- | deter- | deter- | deter- | deter- |
| | mined | mined | mined | mined | mined |
| 13th | 0.8 | 0.91 | 1.54 | 2.2 | 3.07 |

### 3.2 Biofilm Formation

Only the catheter tubing from examples 5 and 6 was tested to detect antimicrobial action and inhibition of biofilm formation on the materials.

TABLE 10
Number of microbes on catheter tubing

| | Number of microbes (CFU/ml) | Number of microbes (CFU/ml) |
|---|---|---|
| | Catheter tubing composed of | Catheter tubing composed of |
| | material of the invention | material comprising |
| Day on which | comprising ciprofloxacin betaine | ciprofloxacin hydrochloride |
| specimen taken | (example 5) | (example 6) |
| 1st | 180 | 0 |
| 2nd | 20 | 0 |
| 3rd | 0 | 0 |
| 4th | 0 | 0 |
| 5th | 0 | 0 |
| 6th | 0 | 10 |
| 7th | 0 | 0 |
| 8th | 0 | 0 |
| 9th | 0 | 1800 |
| 10th | 0 | 400 |
| 11th | 0 | 700 |
| 12th | 0 | 1000 |

Markedly reduced, or no, bacterial colonization was detected for the catheter tubing of the comparative specimen, comprising ciprofloxacin hydrochloride, and also for the catheter tubing of the invention, comprising ciprofloxacin betaine.

### 3.3 Discussion of Results

The dynamic biofilm model permits detection of biofilm formation or detection of inhibition of biofilm formation via the antimicrobial action of a material or of a finished catheter.

The arrangement of the experiment can approximate the natural situation of the catheter in skin.

The factors that can be simulated by the approximation are as follows:
The fluid comprises all of the factors for bacterial growth, corresponding to skin tissue fluid.
The active ingredient can be released slowly from the catheter into the environment, and can develop antimicrobial activity there or directly at the catheter.

Markedly reduced, or no, bacterial colonization was detected for the catheter tubing of the comparative specimen, comprising ciprofloxacin hydrochloride, and also for the catheter tubing of the invention, comprising ciprofloxacin betaine.

The elution profile as a function of time for the catheter tubing exhibits a markedly lower curve for the catheter tubing comprising ciprofloxacin betaine, i.e. this tubing gives markedly less elution of active ingredient over time than the catheter tubing comprising ciprofloxacin hydrochloride. Surprisingly, however, the biofilm studies confirm that, despite the markedly lower level of elution, no colonization of the surface of the catheter tubing is detectable.

It is likewise clear in the case of the extrudate specimens that the elution level depends on the concentration of active ingredient in the material (the higher the content, the higher the level of elution), but that the specimens of the invention give markedly less elution of active ingredient than the comparative samples.

The result of this is that, for the same content of active ingredient, the specimens of the invention can provide a markedly longer period of protection of the surface of the catheter tubing from bacterial colonization, i.e. biofilm formation, because their elution rate is lower.

### Example 18

### Agar Diffusion Test

### 1. Method

The agar diffusion test was used to study antimicrobial action.

### 1.1. Test Plaques

18 ml of NCCLS Mueller-Hinton agar (Merck KGaA Darmstadt/Batch ZC217935 430) were poured into Petri dishes of diameter 9 cm.

### 1.2. Bacterial Suspension

A suspension with density corresponding to McFarland 0.5 in NaCl solution at 0.85% strength was prepared from an overnight culture of test strain of Staphylococcus aureus ATTC 29213 on Columbia blood agar. A “colony pool” composed of from 3 to 4 colonies applied by spotting with an inoculation loop was used for the suspension.

### 1.3. Test Mixture

A sterile absorbent-cotton pad is dipped into the suspension. The excess liquid is expelled under pressure at the edge of the glass. Using the pad, the Mueller-Hinton agar plate is uniformly inoculated in three directions, the angle between each being 60°. Material plaques and test plaques are then placed on the test plate. The test plates were incubated at 37° C. for 24 hours. The antimicrobial action of the specimens was assessed on the basis of zones of inhibition. The smaller plaques stamped out from the injection-molded plaques are used.

TABLE 11
Microbiological activity in the agar diffusion test
with respect to Staphylococcus aureus ATTC 29213

| | S. aureus Zone of | | Concentration |
|---|---|---|---|
| Test strain | inhibition: diameter | Active ingredient | [%] |
| Material | 29213 | | |
| Example 7 | 12 | Ciprofloxacin | 0.25 |
| (comparative example) | | hydrochloride | |
| Example 8 | 14 | Ciprofloxacin | 0.5 |
| (comparative example) | | hydrochloride | |
| Example 9 | 14 | Ciprofloxacin | 1.0 |
| (comparative example) | | hydrochloride | |
| Example 10 | 20* | Ciprofloxacin | 1.5 |
| (comparative example) | | hydrochloride | |
| Example 11 | 20* | Ciprofloxacin | 2.0 |
| (comparative example) | | hydrochloride | |
| Example 12 | 6 | Ciprofloxacin | 0.25 |
| | | (betaine) | |
| Example 13 | 8 | Ciprofloxacin | 0.5 |
| | | (betaine) | |
| Example 14 | 12 | Ciprofloxacin | 1.0 |
| | | (betaine) | |
| Example 15 | 20* | Ciprofloxacin | 1.5 |
| | | (betaine) | |
| Example 16 | 20* | Ciprofloxacin | 2.0 |
| | | (betaine) | |

*The agar diffusion test is not capable of differentiating between the different concentrations. An increase in the amount eluted gives no further acceleration in diffusion in the agar, and the same zone of inhibition is therefore observed for these concentrations.

The inhibition zones of the specimens from examples 12 to 14 of the invention are smaller than those of the comparative specimens from examples 7 to 9. The zones of inhibition can be used to draw conclusions concerning the intensity or quantity of the active ingredients released, when the specimens of materials are compared. This confirms the results from the elution profiles.

### Example 19

Pieces of length about 1 mm were removed by cutting from the catheter tubing from example 5 (of the invention) and 6 (comparative example), in each case at intervals of about 1 cm.

Test plates were prepared as described in example 18, the agar diffusion test. The cut surfaces of the catheter tubing sections were placed on the agar plates. The treatment of the test mixture then continued as in example 18.

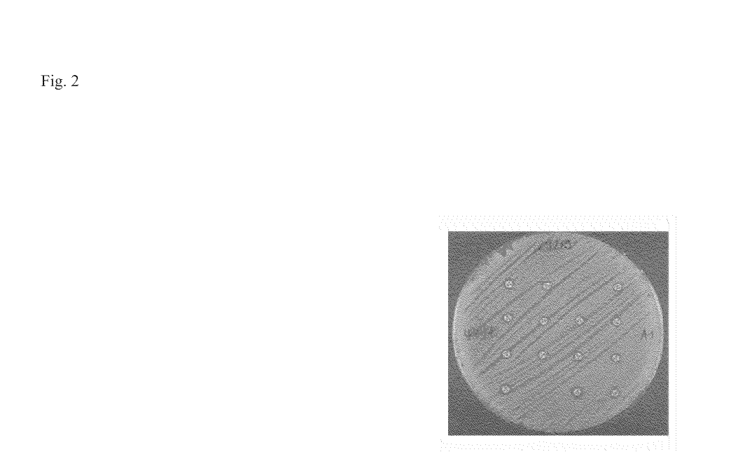

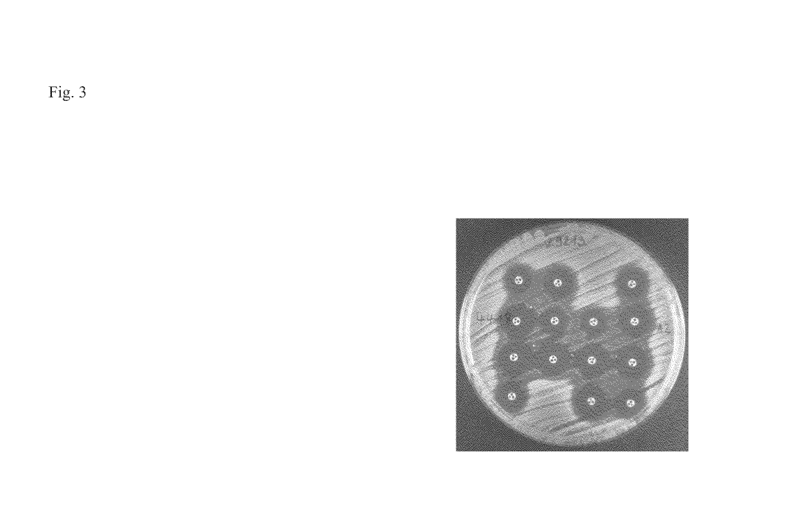

FIGS. 2 and 3 respectively show agar plates colonized by Staphylococcus aureas ATTC 29213. A zone of inhibition has formed around the superposed catheter tubing sections. FIG. 2: sections of catheter tubing from example 5 (of the invention); FIG. 3: sections of catheter tubing from example 6 (comparative example).

The agar diffusion test reveals that all of the catheter tubing gives a zone of inhibition and exhibits antibacterial activity. At identical concentration, less active ingredient is eluted in the case of the catheter tubing of the invention from example 5 than in the case of catheter tubing from example 6. The catheter tubing of the invention therefore remains protected against biofilm formation for a markedly longer time. The fact that the diameter of the zone of inhibition of all of the specimens on a plate is the same moreover clearly shows that, for both varieties of tubing, distribution of the active ingredient is homogeneous across the entire length of the catheter tubing.

1. A molding composition comprising at least one thermoplastically processable polymer, and at least one partially neutralized active ingredient with antibacterial, antiprotozoic, and/or antimycotic activity.

2. The molding composition as claimed in claim 1, wherein the partially neutralized active ingredient is an active ingredient having basic functionality, and wherein said basic functionality has been partially neutralized with an acid.

3. The molding composition as claimed in claim 1, wherein the partially neutralized active ingredient is an active ingredient with acidic functionality, and wherein said acidic functionality has been partially neutralized with a base.

4. The molding composition as claimed in claim 1, wherein the partially neutralized active ingredient is an ingredient having betaine structure or having zwitterion structure, and has been partially neutralized with a base or acid.

5. The molding composition as claimed in claim 1, wherein one equivalent of basic functionality in the active ingredient has been partially neutralized with from 0.01 to 0.95 equivalent of acid, or one equivalent of acidic functionality in the active ingredient has been partially neutralized with from 0.01 to 0.95 equivalent of base.

6. The molding composition as claimed in claim 1, wherein the thermoplastically processable polymer is selected from the group consisting of thermoplastic polyurethanes, polyether block amides, and copolyesters.

7. The molding composition as claimed in claim 1, wherein the active ingredient is ciprofloxacin.

8. The molding composition as claimed in claim 1 wherein the partially neutralized active ingredient is ciprofloxacin partially neutralized with hydrogen chloride.

9. The molding composition as claimed in claim 1, wherein the partially neutralized active ingredient is used in the concentration range, in the form of non-neutralized active ingredient, of from 0.5 to 2.0% by weight, based on the molding composition.

10. A method for the production of a molding comprising using a molding composition of claim 1 to form said molding.

11. A medical product comprising a molding composition as claimed in claim 1.

12. A method of claim 10, wherein said molding comprises a medical product.

13. A method of claim 12, wherein said medical product comprises a central venous catheter, urocatheter, flexible tubing, shunt, cannula, connector, stopper, and/or distributor valve.

14. A medical product of claim 11 comprising central a venous catheter, urocatheter, flexible tubing, shunt, cannula, connector, stopper, and/or distributor valve.

15. The molding composition as claimed in claim 2, wherein the active ingredient is ciprofloxacin.

16. The molding composition as claimed in claim 3, wherein the active ingredient is ciprofloxacin.

17. The molding composition as claimed in claim 2, wherein the partially neutralized active ingredient is ciprofloxacin partially neutralized with hydrogen chloride.

18. The molding composition as claimed in claim 2, wherein the partially neutralized active ingredient is used in the concentration range, in the form of non-neutralized active ingredient, of from 0.5 to 2.0% by weight, based on the molding composition.

19. The molding composition as claimed in claim 3, wherein the partially neutralized active ingredient is ciprofloxacin partially neutralized with hydrogen chloride.

20. The molding composition as claimed in claim 3, wherein the partially neutralized active ingredient is used in the concentration range, in the form of non-neutralized active ingredient, of from 0.5 to 2.0% by weight, based on the molding composition.
